(19) United States
(12) Patent Application Publication
(10) Pub. No.: US 2012/0245479 A1
(43) Pub. Date: Sep. 27, 2012

(54) Physiology Monitoring and Alerting System and Process

(76) Inventors: Meena Ganesh, Clifton Park, NY (US); Jeffrey Michael Ashe, Gloversville, NY (US); Lijie Yu, Clifton Park, NY (US); Catherine Mary Graichen, Malta, NY (US)

(21) Appl. No.: 13/069,483

(22) Filed: Mar. 23, 2011

(51) Int. Cl. A61B 5/02; A61B 5/08

(52) U.S. Cl. 600/508; 600/529

(57) ABSTRACT

A system for monitoring physiology, having: a RADAR transmitter and a RADAR receiver; a state estimation module configured to process a returned RADAR signal to detect a presence of motion and set a motion state upon said presence of motion, said state estimation module configured to detect a presence of one or more physiological parameters including heartbeat and respiration, and said state estimation module configured to assign a still state or a concern state based on said presence of physiological parameters; a rate estimation module configured to estimate one or more estimated physiological rates including an estimated respiration rate and an estimated heart rate; and an alerting module configured to provide an alert if an alert value exceeds an alert value threshold, wherein the alert value is derived from at least one of the motion state, concern state, still state and the estimated physiological rates.

### STATEMENT REGARDING FEDERALLY SPONSORED DEVELOPMENT

This invention was made with government support under Contract No. 2007-DE-BX-K176, awarded by the United States Department of Justice. The United States Government has certain rights in the invention.

### BACKGROUND

Radio Detection and Ranging (RADAR) provides for object identification by using radio waves. It is primarily known for identifying parameters such as speed, direction, range, and altitude of planes, ships and automobiles. The typical method of operation includes a transmitter that transmits the radio waves, generally from some form of antenna, wherein a certain portion of the radio waves are reflected from an object. The reflected waves are then processed to acquire the desired properties of the object. There are a wide array of applications and implementations using RADAR.

RADAR systems are also capable of monitoring human physiological attributes such as heartbeat and respiration. This monitoring thereby permits unobtrusive monitoring of a person's physiology, and likewise, state of health. However, accurately measuring the movement from the pulsations resulting from heartbeat and breathing using RADAR has conventionally required relatively sophisticated and complex RADAR equipment, since such movements are relatively small. Such sophisticated RADAR equipment is typically expensive for use in the applications where RADAR monitoring of a person's physiology would provide benefit. Furthermore, the processing of the data has a number of attributes that make the it challenging. Consequently, there remains a need in the art for an inexpensive and relatively less complex physiology monitoring RADAR system.

### BRIEF DESCRIPTION

One embodiment of the present system is for monitoring physiology, and comprises: a RADAR apparatus comprising a RADAR transmitter configured to deliver a RADAR signal to a subject, and a RADAR receiver configured to receive a returned RADAR signal from the subject; a state estimation module configured to process the returned signal to detect a presence of motion and set a motion state upon said presence of motion, said state estimation module configured to detect a presence of one or more physiological parameters, said physiological parameters comprising at least one of heartbeat and respiration, and said state estimation module configured to assign a still state or a concern state based on said presence of physiological parameters; a rate estimation module configured to process the returned signal and estimate one or more estimated physiological rates comprising at least one of an estimated respiration rate and an estimated heart rate; and an alerting module configured to provide an alert if an alert value exceeds an alert value threshold, wherein the alert value is derived from at least one of the motion state, concern state, still state and the estimated physiological rates.

Another embodiment of the present system is for monitoring physiology, and comprises: a RADAR apparatus comprising a RADAR transmitter configured to deliver a RADAR signal to a subject, and a RADAR receiver configured to receive a returned RADAR signal from the subject, wherein the returned signal comprises at least a first signal and a second signal, each having different signal characteristics; a state estimation module configured to process at least the first signal and the second signal to detect a presence of motion and one or more physiological parameters, said physiological parameters comprising at least one of heartbeat and respiration, wherein the state estimation module is configured to assign a state estimation state based on the presence of motion, the physiological parameters, and combinations thereof; a rate estimation module configured to further process at least the first signal and the second signal and provide estimated physiological rates comprising at least one of a heart rate and a respiration rate; and an alerting module configured to set an alert value and communicate an alert based on the alert value, wherein the alert value is derived from processing from the state estimation module and the rate estimation module.

A further embodiment provides a method for monitoring physiology, comprising: A method for monitoring physiology, comprising: providing a RADAR transmitter to deliver a RADAR signal to a subject, and a RADAR receiver to receive a returned RADAR signal from the subject; processing the returned RADAR signal to detect a presence of motion; based upon the presence of motion, further processing the returned RADAR signal to determine a presence of physiological parameters, said physiological parameters comprising at least one of heartbeat and respiration; based upon the presence of the physiological parameters, processing estimated physiological rates from the returned RADAR signal, said estimated physiological rates comprising at least one of a heart rate and a respiration rate; and setting an alert based on at least one of the presence of motion, the presence of physiological parameters and the estimated physiological rates.

### BRIEF DESCRIPTION OF THE DRAWINGS

These and other features, aspects, and advantages of the present systems and techniques will become better understood when the following detailed description is read with reference to the accompanying drawings in which like characters represent like parts throughout the drawings, wherein:

FIG. 1A shows a schematic perspective of a RADAR based physiology monitoring system in accordance with one embodiment.

FIG. 1B shows a schematic perspective of the state estimation module, rate estimation module, and alerting module of the RADAR based physiology monitoring system of FIG. 1A in accordance with one embodiment.

FIG. 1C shows a schematic perspective of the state estimation module of FIG. 1B in one embodiment.

FIG. 2A is a diagrammatic perspective of the physiological monitoring techniques in accordance with one embodiment.

FIG. 2B is a flowchart showing an embodiment of state estimation logic.

FIG. 3 is a flowchart showing another embodiment of state estimation logic.

FIG. 4 is a flowchart showing yet another embodiment of state estimation logic.

FIG. 5 is a flowchart showing an embodiment of the heart rate and respiration rate estimation logic.

FIG. 6 is a flowchart showing an alternate embodiment of the heart rate and respiration rate estimation logic.

FIG. 7 is a flowchart showing one embodiment of alerting logic.

### DETAILED DESCRIPTION

The present disclosure describes systems and techniques employing RADAR hardware together with software that permits physiological monitoring of subjects.

Many environments and applications exist where a low cost, unobtrusive physiology monitoring system provide benefit. For example, such a system would enable correctional institution staff to monitor inmate physiology. Information gathered from the system could alert prison staff to potential health problems, including suicide attempts. Other examples include physiological monitoring of patients at nursing homes, hospitals and care facilities. A further example relates to physiological monitoring of a person at home. However, RADAR units sophisticated enough to perform such monitoring have historically been too expensive for such institutions to afford.

The present monitoring system in one embodiment can be viewed in terms of the hardware employed, and the processing performed to the signal returned to the RADAR receiver and transmitted to a computer processor.

The processing according to another embodiment can be viewed as a collection of interrelated processing modules. In one example there are three modules, including state estimation; rate estimation; and alerting. In one example, the state estimation module attempts to ascertain if a person is exhibiting signs associated with a state of good health. If it cannot ascertain this state with an acceptable level of confidence, it turns to the rate estimation module for further information that it will use to make a final decision as to the state of the subject. The results of the state assessment are used by an alerting module to determine if the subject requires attention.

State estimation processing by way of one example seeks to determine if a subject is performing gross body movements. Such movements are those greater than movements resulting from heartbeat pulsations and respiration chest movements. Some examples of gross body movement include walking, shaking a leg, turning over in bed, and coughing which are considered such gross body motions, herein referred to as motion. The gross body movement is generally any movement that impacts the processing of the heartbeat and respiration movements.

In one example, if any one of the state estimation predictors ascertains an indication of motion, it assumes there is motion and sets the subject's state to motion. If all or most of the various state estimation predictors indicate a lack of motion, the state estimation module sets the state to concern. Any other scenarios result in the state being set to still or concern. When not set to motion or concern, the subject could be in any of multiple states, ranging from good health states such as sleeping, to bad health states such as low or no heartbeat or respiration. When not set to motion or concern, the state estimation module turns to the rate estimation software for an estimation of the subject's heart rate and respiration rate. Once the heart rate and respiration rate are estimated, they are compared to acceptable heart rates and respiration rates. The acceptable heart rates and respiration rates in one example are based on the particulars of the subject and the circumstances. For example, a range can be determined based on historical data of the personal parameters of the subject such as age, gender, and similar aspects. The range can also be personalized to a specific subject and known historical personal data. If both the heart rate and respiration rate are within the acceptable range, then the system estimates the subject is in a good health state and, for example, the state may be set to still. If both the heart rate and respiration rate are outside their respective acceptable range, the system assumes the subject is in a bad health state and the state is set to concern. The system can set the state to still or concern if certain estimates are within an acceptable range but not certain others. The rate estimation processing is typically more reliable during times when there are no gross body motions occurring. While the rate estimation module will typically return rate information during times of motion, the results may be inaccurate and are therefore excluded or minimized. In one example, rate estimation is limited to those times when the state module ascertains that there is no or little motion.

In one embodiment, alerting tracks the state setting and determines if an alert is necessary. Various alerting algorithms can be used to determine whether an alert is warranted. One example is where a count is maintained for the estimated parameter such as state. Each state is assigned its own adjustment value, and the count is adjusted by the adjustment value assigned for the state. The count can have a minimum and/or maximum value and a threshold, and if the count exceeds the threshold, an alert is indicated.

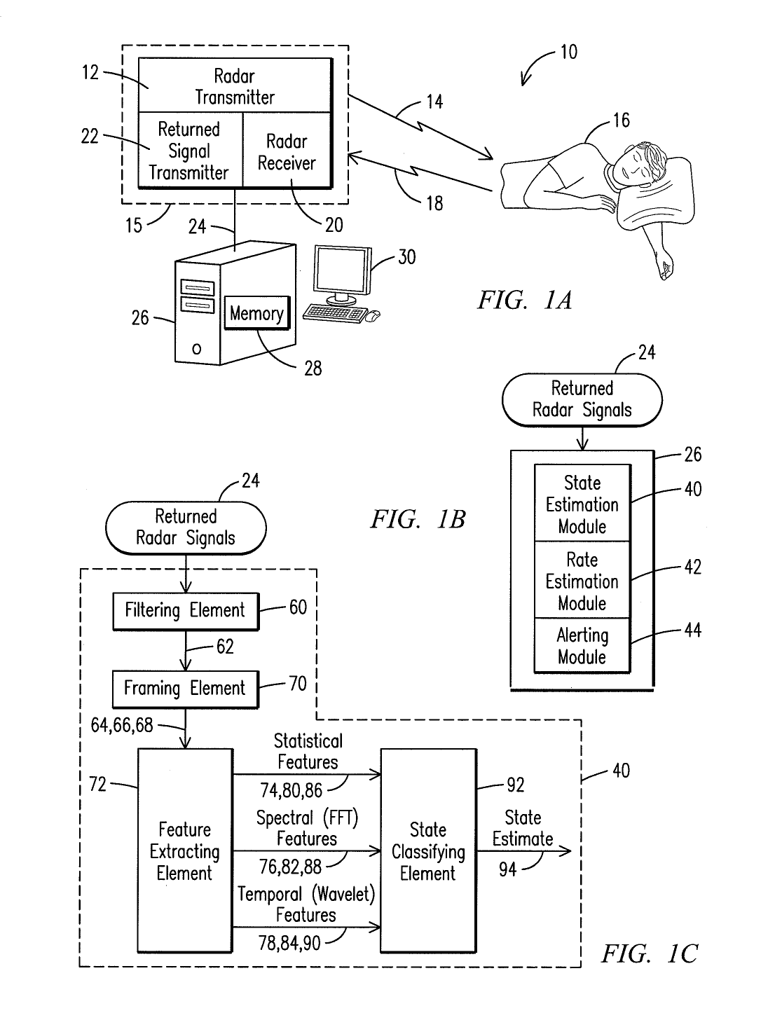

Referring to the figures, FIG. 1A shows one example of the physiology monitoring and alerting system 10. In one embodiment the alerting system 10 includes a RADAR unit 15 that encompasses a RADAR receiver 20, RADAR transmitter 12 and returned signal transmitter 22. The RADAR transmitter 12 transmits an outbound RADAR signal 14 to a subject 16. The outbound RADAR signal 14 is reflected off the subject 16 as a reflected RADAR signal 18. The reflected RADAR signal 18 is received by a RADAR receiver 20. In one embodiment the RADAR transmitter 12 and RADAR receiver 20 are components of a single, portable hardware device and can be deployed as a transceiver. For example, they may be housed in hardware dedicated to the physiology monitoring and alerting system 10, or they may be incorporated into hardware as an additional feature, such as a cell phone, a Personal Digital Assistant (PDA) or other portable electronic device. However, the RADAR transmitter 12 and the RADAR receiver 20 can be discrete components as well. In one application the subject can be sleeping and the system 10 can monitor for health conditions such as sleep apnea or sudden infant death syndrome. It should be readily understood that the subject 16 can be any subject and not merely a sleeping subject. For example, the physiological parameters of a person sitting in an airport can be monitored to check for high pulse rate as an indicator of a security risk.

The RADAR receiver 20 may perform a variety of operations on the reflected RADAR signal 18 including filtering, amplification, downconversion and/or demodulation, and analog-digital conversion before the returned signal 24 is transmitted via a returned signal transmitter 22 along a returned signal transmission path to a processor 26. The returned signal transmission path may be a hard line, or wireless path of any sort sufficient to carry the returned signal 24. The returned signal transmitter 22 in one example processes and packages the returned signal 24 prior to transmission to the processor 26. The processor 26, such as a microprocessor or other computing device typically includes some form of memory 28 used by the processor 26. The memory 28 may store among other things, returned RADAR signals, historic records of returned RADAR signals as well as the software and modules necessary for processing. In one example, information about the subject's 16 motion and physiology can be obtained by processing the returned signal 24 using the processor 26 and memory 28. Physiology refers to physiological parameters, such as the presence of a heartbeat and/or the presence of respiration, as well as physiological rates, such as the rate at which a heart is beating (heart rate) and/or the rate at which one is breathing (respiration rate). The presence of physiological parameters in one example refers to some indication of such respiration and/or heartbeat that is based on threshold levels. In this embodiment, the returned signals 24 are transmitted to a processor 26 to ascertain physiological features such as respiration and heartbeat. In one example the returned signal 24 transmitted by the returned signal transmitter 22 is processed by a state estimation module and rate estimation module. While the processor 26 is depicted as separate from the RADAR receiver, a further embodiment incorporates a processor 26 with the RADAR transmitter 12 and RADAR receiver 20, thereby eliminating the returned signal transmitter 22.

In one embodiment the processor 26 includes various communication and alerting mechanisms. Coupled to the processor 26 in one example is a user interface 30 that allows an operator to interface with the processor and thereby dynamically alter system parameters, modify thresholds, or otherwise interface with the processor 26 and memory 28.

FIG. 1B shows a state estimation module 40, a rate estimation module 42, and an alerting module 44 within the processor 26. The state estimation module 40 in one example determines a subject's state as either motion, still, or concern (i.e. “state estimation”). The rate estimation module 42 in one example estimates physiology of a subject, such as heart rate and respiration rate (i.e. “rate estimation”). The alerting module 44 in one example tracks estimated states and determines if an alert is necessary (i.e. alerting).

FIG. 1C shows a more detailed schematic of elements of the state estimation module 40 (i.e. state estimation) according to one example. The returned signal 24 from the RADAR unit 15 is received by the processor 26 such as shown in FIG. 1A. This signal may be digital or it may be analog and subsequently converted to digital. In the case of an analog signal, traditionally high sample rates associated with PC based data acquisition systems, such as 5 kHz, can be decimated to sample rates on the order of 40 Hz to 200 Hz. In one example, the returned signal 24 is fed into a filtering element 60 that creates a signal frame 62 of a limited duration from the returned signal 24.

The signal frame 62 can include a number of frequency bands that can be distinguished based upon the frequency band characteristics. According to one embodiment, a motion frame 64, a heartbeat frame 66, and a respiration frame 68 are extracted in a framing element 70 and are distinguished based upon the frequency band characteristics. Frequencies responsive to the presence of motion typically range from about 4 Hz up to about 10 Hz. Frequencies responsive to the presence of a heartbeat typically range from about 1 Hz to about 3 Hz. Frequencies responsive to the presence of respiration typically range from just above 0 Hz (DC) to about 1 Hz. Consequently, the returned signal 24 should generally cover at least those frequencies. In one example, the motion, or high band, frame will be of a signal comprising from about 4 Hz up to about 10 Hz, the heartbeat, or mid band, frame will be of a signal comprising from about 1 Hz to about 3 Hz, and the respiration, or low band, frame will be of a signal comprising from just above 0 Hz (DC) to about 1 Hz. Low pass and band pass filters may be utilized to extract the motion frames, heartbeat frames and respiration frames from each signal frame.

In one embodiment, once each of the frames 64, 66, 68 is generated, features are extracted from each frame 64, 66, 68 in a feature extracting element 72. For example, features associated with the motion frame 64 are extracted as motion features. Likewise, features associated with the heartbeat frame 66 are extracted as heartbeat features, and features associated with the respiration frame 68 are extracted as respiration features. Features can be, for example, statistical features, spectral features, or temporal features. Thus, for motion frames, motion statistical features 74, motion spectral features 76, and motion temporal features 78 are extracted. For heartbeat frames heartbeat statistical features 80, heartbeat spectral features 82, and heartbeat temporal features 84 are extracted. For respiration frames, respiration statistical features 86, respiration spectral features 88, and respiration temporal features 90 are extracted.

Thus in one example, the returned signal 24 is filtered into the low, mid and high band signals. These filtered low, mid and high band signals are then processed by the framing element 70. In the framing element 70, frames for each of the low mid and high signals are selected. These can be called frames or windows. The frames can be different lengths depending on the feature to be detected. State estimation frames may be, for example, on the order of 5 seconds, heart rate frames on the order of 10 seconds and respiration frames on the order of 30 seconds.

Statistical features in one example include mean, variance, higher order moments, and kurtosis for the frame. Spectral features may be based on Fast Fourier Transform (FFT) or similar spectral techniques. Spectral features in one example may be the frequencies of the FFT bins containing the top highest signal amplitudes. Temporal features may be based on wavelet transforms, and in one example may include the wavelet coefficient, slope of wavelet coefficient change etc. For example, a continuous wavelet transform may be used, and has been found to be useful for determining still states when a subject is holding his breath. A stationary wavelet transform may be used and has been found to be useful for determining still states when a subject is shallow breathing.

Once the features 74, 76, 78, 80, 82, 84, 86, 88, 90 for each frame are extracted from the feature extracting element 72, the features are processed in a state classifying element 92 which estimates a state 94. In an embodiment, features are compared to known feature sets. For example, a database may contain known motion feature sets taken during times when a subject is known to be moving. The extracted motion frame features 74, 76, 78 may be compared to features known to be indicative of motion, and a determination made as to whether the extracted features match known motion feature sets. Techniques such as principal component analysis may be used for clustering of features. How closely the extracted feature set matches the known motion feature set(s) may vary; it may be set in advance, or it may be adjusted over time when learning algorithms are employed to make the match assessment. It is also possible to permit a user to adjust sensitivity based on attributes such as observations. Likewise, heartbeat features 80, 82, 84 and respiration features 86, 88, 90 may be compared to known respective feature sets.

Once features are extracted from each frame, the state classifying element 92 will employ an algorithm to determine if it has enough information to base a state decision 94. The state classifying element 92 in one embodiment checks if there is sufficient motion (i.e. if the motion frame features 74, 76, 78 sufficiently indicate motion) and if so may set the state to ‘motion’. If the state classifying element 92 identifies motion it may set the state to motion regardless of what the heartbeat frame features 80, 82, 84 and respiration frame features 86, 88, 90 indicate. In other words, the state classifying element 92 favors any estimation of motion. If the motion frame features 74, 76, 78 indicate a lack of motion, the heartbeat frame features 80, 82, 84 indicate a lack of heartbeat, and the respiration frame features 86, 88, 90 indicate a lack of respiration then the state classifying element 92 sets the state to ‘concern’. In other words, the state classifying element 92 may disfavor an estimation of concern, such that all of the frames must unanimously indicate and lack of motion, heartbeat or respiration. Otherwise, if the motion frame features 74, 76, 78 indicate a lack of motion but any of the heartbeat frame features 80, 82, 84 indicate a heartbeat or any of the respiration frame features 86, 88, 90 indicate respiration the state classifying element 92 may set the state to ‘still’ or ‘concern’ depending on rate estimation.

As detailed herein, the rate estimation module in one example estimates a heart rate and a respiration rate which is returned to the state classifying element 92. The heart rate and respiration rate are compared in the state classifying element 92 to acceptable heart rate and respiration rates. In one example the state classifying element 92 then passes to the alerting module 44 whether the estimation is “motion”, “concern”, “still but with heart and respiration rates within the acceptable range”, (‘still’), or “still with either one or both of the heart or respiration rates outside the acceptable range”, (‘concern’). The range of acceptable heart rate and respiration rate can be predetermined such as based on some historical data, can be adjusted by an operator, or can be set by an algorithm that learns the subject being monitored.

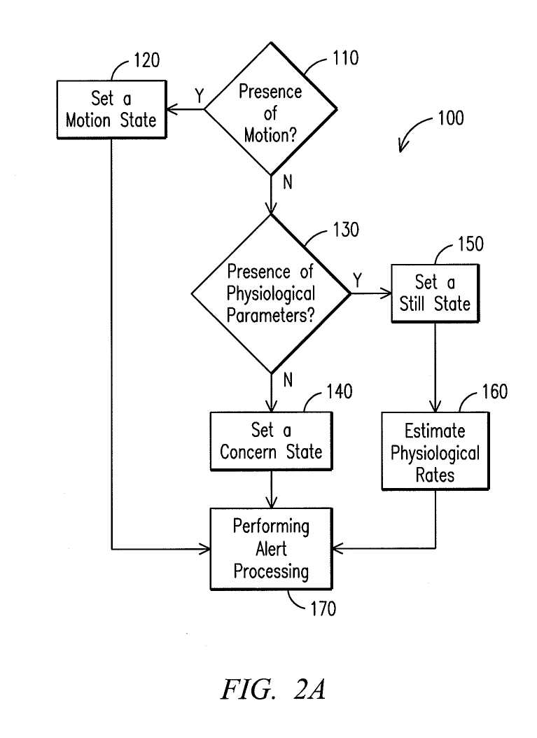

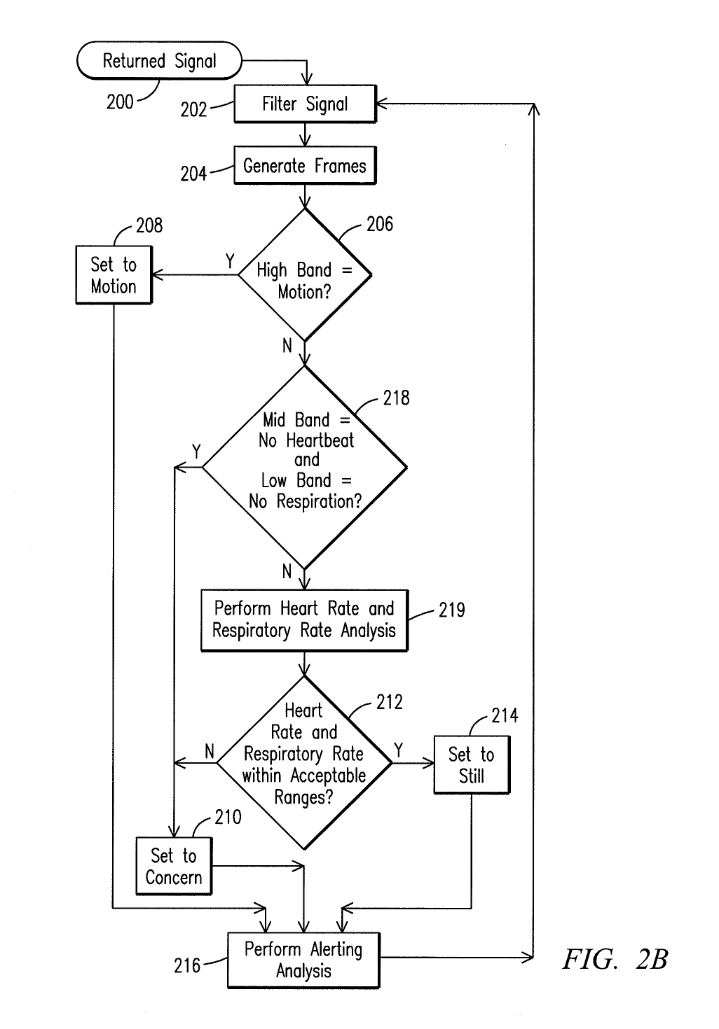

Referring to FIG. 2A, a simplified flow chart presentation of the processing 100 according to one embodiment is depicted. According to this embodiment, the first step in the processing is to detect a presence of motion 110 from the returned RADAR signals. The state estimation module as shown in FIG. 1B is an example of the hardware and software components that are configured to detect the presence of motion 110. If there is a presence of motion, a motion state is set 120 and in this example the information concerning the motion state is subject to alert processing 170.

In one example, if there is no presence of motion 110 and the motion state is not set, the returned RADAR signals are processed to detect the presence of physiological parameters 130. In another example, processing for the presence of physiological parameters 130 are continuously or periodically processed regardless of the motion state but are only evaluated when there is no presence of motion. In one example the physiological parameters include features such as a heartbeat and/or respiration. If there is no presence of physiological parameters 130, the process is configured to set a concern state 140 since this may indicate a potential medical condition. The concern state 140 in this example is then subject to alert processing 170. In one example, if there is a presence of all or at least one of the physiological parameters 130, the processing in one embodiment sets a still state 150 and the returned RADAR signals are processed to estimate physiological rates 160. In a more conservative example, if there is only one of the physiological parameters 130, the process is configured to set a concern state. The physiological rate estimates in one example include heart rate and/or respiration rate. The physiological rate estimates 160 are made available for the alert processing 170 in order to establish the appropriate alert condition.

FIG. 2B shows one embodiment of the state estimation process of the state estimation module 40 of FIG. 1C in flow chart form. The returned signal 200 from the RADAR unit is received by the processor such as shown in FIG. 1A. The returned signal 200 is fed into a filter that samples a signal frame of a limited duration from the returned signal during a filtering step 202. According to one embodiment, a motion frame, a heartbeat frame, and a respiration frame are extracted in a frame generating step 204. As above, once each frame is generated, features are extracted from each frame. Once the features for each frame are extracted, the state estimation in one embodiment checks if there is sufficient high band motion in a motion assessment step 206 and may set the state to ‘motion’ 208 if the motion features sufficiently indicate motion. In one example if the motion features indicate a lack of motion, the state estimation checks if there is insufficient mid-band and low-band motion in a mid-band and low-band assessment step 218 and may set the state to ‘concern’ 210 if the mid-band and low-band features indicate a lack of heartbeat and a lack of respiration. Thus in this example all three frames should indicate a lack of motion, heartbeat, and respiration in order for the state estimation to set the state to concern. Otherwise the state estimation determines it needs more information before making a decision, in which case it performs rate estimation analysis 219 for detailed physiological estimates. The heart rate and respiration rate returned by the rate estimation analysis 219 are compared to acceptable heart rate and respiration rates in a heart rate and respiration rate comparison step 212. If the heart rate and respiration rate estimates are within acceptable ranges, the state estimation sets the state to ‘still’ 214 otherwise the state is set to ‘concern’ 210. The state estimation then performs alerting analysis 216 whether the state estimation is motion, concern, still but with heart and respiration rates within the acceptable range, or still with either one or both of the heart or respiration rates outside the acceptable range. It is important to note that there is a great deal of flexibility in the system in setting the state to ‘still’ 214 or concern ‘210’. For example, indication of a heartbeat, indication of respiration, or indication of both may be checked for in alternate embodiments. Further, if both are checked for, only one or both may be required to be indicated in order to set the state to ‘still’ 214. Also, a heart rate, a respiration rate, or both may be estimated. If both are estimated, only one or both may be required to be within a respective acceptable range in order to set the state to ‘still’ 214.

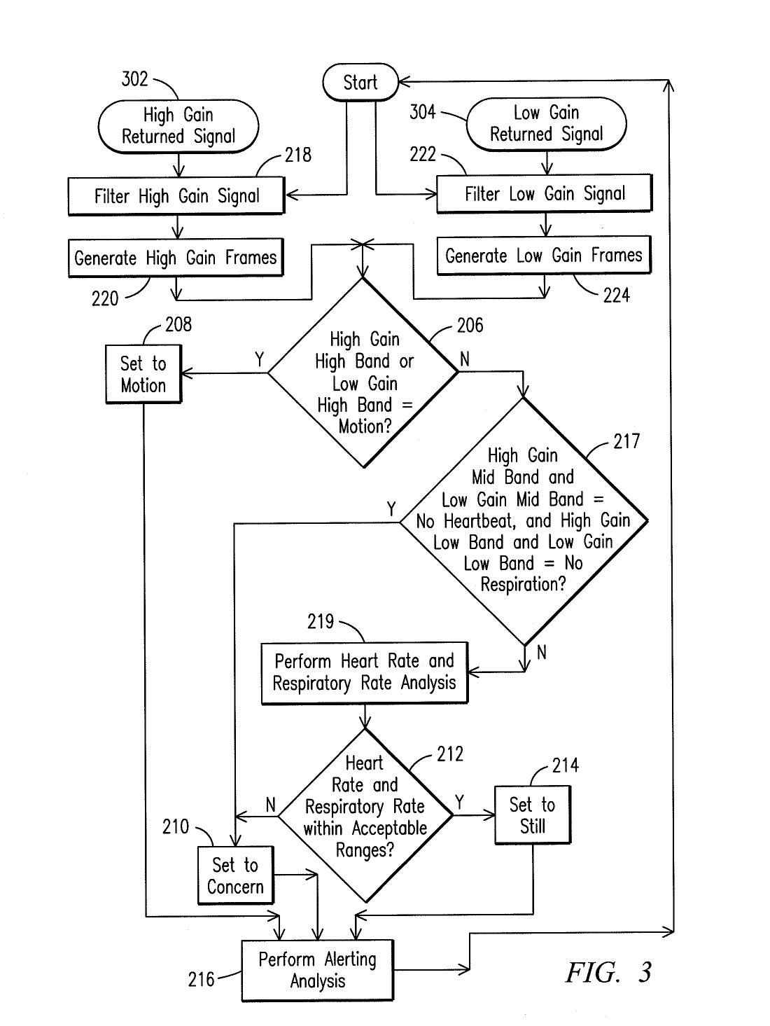

In one embodiment the returned signal may comprise signals having different gain characteristics. For example, the returned signal in one example has a high gain returned signal 302 and a low gain returned signal 304. In that case each gain is processed individually for a state estimation as can be seen in FIG. 3. The high gain returned signal 302 is filtered in a high gain filtering step 218, and high gain signal frames are generated in a high gain signal frame generation step 220. The low gain signal 304 is filtered in a low gain filtering step 222, and low gain signal frames are generated in a low gain filtering step 224. If either high gain high band signal or low gain high band signal results in an estimate of motion, the state is set to motion 208 in a high band motion assessment step 206. If both the high gain high band signal or low gain high band signal does not result in an estimate of motion, the high gain low band, high gain mid band, low gain low band and low gain mid band are evaluated for heartbeat and respiration in a mid-band and low-band assessment step 217. If the high gain low band, high gain mid band, low gain low band and low gain mid band features indicate a lack of heartbeat or a lack of respiration, the state is set to ‘concern’ 210. Otherwise the state estimation determines it needs more information before making a decision, in which case it performs rate estimation analysis 219 for detailed physiological estimates. The heart rate and respiration rate returned by the rate estimation analysis 219 are compared to acceptable heart rate and respiration rates in a heart rate and respiration rate comparison step 212. If the heart rate and respiration rate estimates are within acceptable ranges, the state estimation sets the state to ‘still’ 214 otherwise the state is set to ‘concern’ 210. The state estimation then performs alerting analysis 216 whether the state estimation is motion, concern, still but with heart and respiration rates within the acceptable range, or still with either one or both of the heart or respiration rates outside the acceptable range.

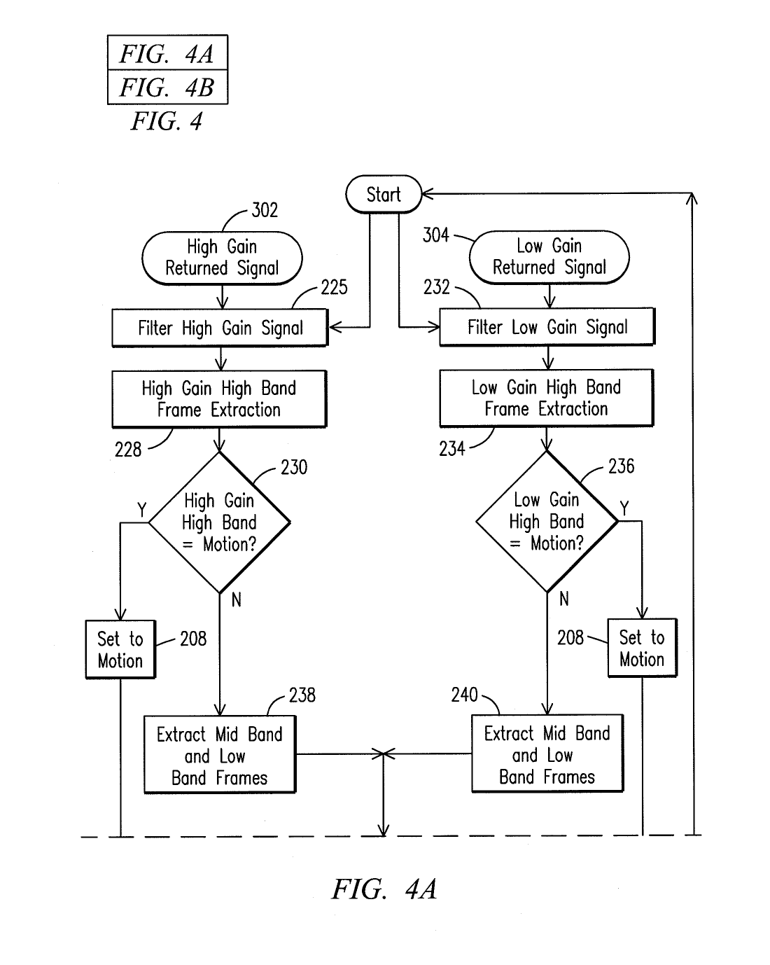

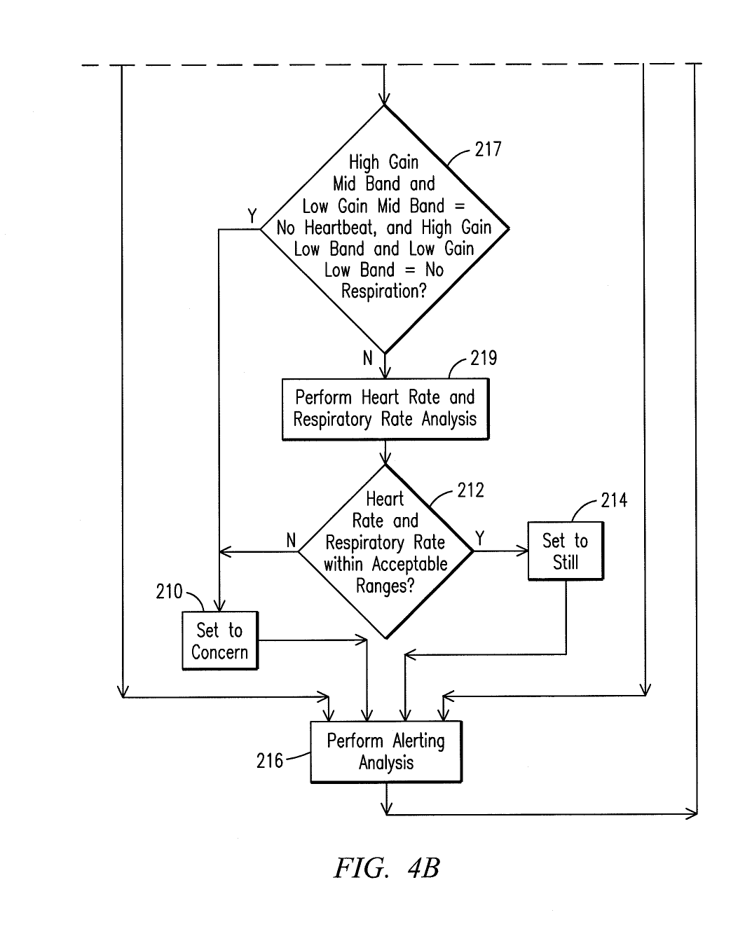

In another embodiment shown in FIG. 4 the returned signal comprises a high gain returned signal 302 and a low gain returned signal 304. Instead of extracting all three frames, such as extracting motion, heartbeat, and respiration frames from each signal frame and then extracting features from each frame before checking the motion frame for the presence of motion, the state estimation may first check for motion only. For example, the high gain signal may be filtered in a high gain filtering step 225, a high gain high band frame extracted in a high gain high band frame extraction step 228, and the high gain high band frame checked for motion in a high gain high band motion check step 230. If motion is indicated, the state is set to motion 208 and alerting analysis is performed 216 wherein the process is repeated without processing further frames. The low gain signal may also be filtered in a low gain filtering step 232, a low gain high band frame extracted in a low gain high band frame extraction step 234, and the low gain high band frame checked for motion in a low gain high band motion check step 236. If motion is indicated, the state is set to motion 208 and the alerting analysis is performed 216 wherein the process is repeated without processing further frames. Thus, if either the high gain high band frame or the low gain high band frame indicates motion, the state is set to motion 208 and no further state estimation processing is performed. As a result the demand on processing resources may be decreased. If during both the high gain high band check 230 there is no motion and during the low gain high band check 236 there is no motion, the high gain mid band and high gain low band frames are extracted 238 and the low gain mid band and low gain low band frames are extracted 240. The high gain low band, high gain mid band, low gain low band and low gain mid band are evaluated for a presence of a heartbeat and respiration in a mid-band and low-band assessment step 217. If the high gain low band, high gain mid band, low gain low band and low gain mid band features indicate a lack of heartbeat or a lack of respiration, the state may be set to ‘concern’ 210. Otherwise the state estimation determines it needs more information before making a decision, in which case it performs rate estimation analysis 219 for detailed physiological estimates. The heart rate and respiration rate returned by the rate estimation analysis 219 are compared to acceptable heart rate and respiration rates in a heart rate and respiration rate comparison step 212. If the heart rate and respiration rate estimates are within acceptable ranges, the state estimation sets the state to ‘still’ 214 otherwise the state is set to ‘concern’ 210. It is important to note that as in the single signal embodiment, there is a great deal of flexibility in the system in setting the state to ‘still’ 214 or concern ‘210’. For example, indication of a heartbeat, indication of respiration, or indication of both may be checked for, and this may occur for one gain or both in alternate embodiments. Further, if both are checked for, only one or both may be required to be indicated in order to set the state to ‘still’ 214, and the different signals may or may not be required to agree with each other. Also, a heart rate, a respiration rate, or both may be estimated. If both are estimated, only one or both may be required to be within a respective acceptable range in order to set the state to ‘still’ 214. This may occur for one or both gains, and both gains may or may not be required to agree with each other.

The state estimation then performs alerting analysis 216 whether the state estimation is motion, concern, still but with heart and respiration rates within the acceptable range, or still with either one or both of the heart or respiration rates outside the acceptable range.

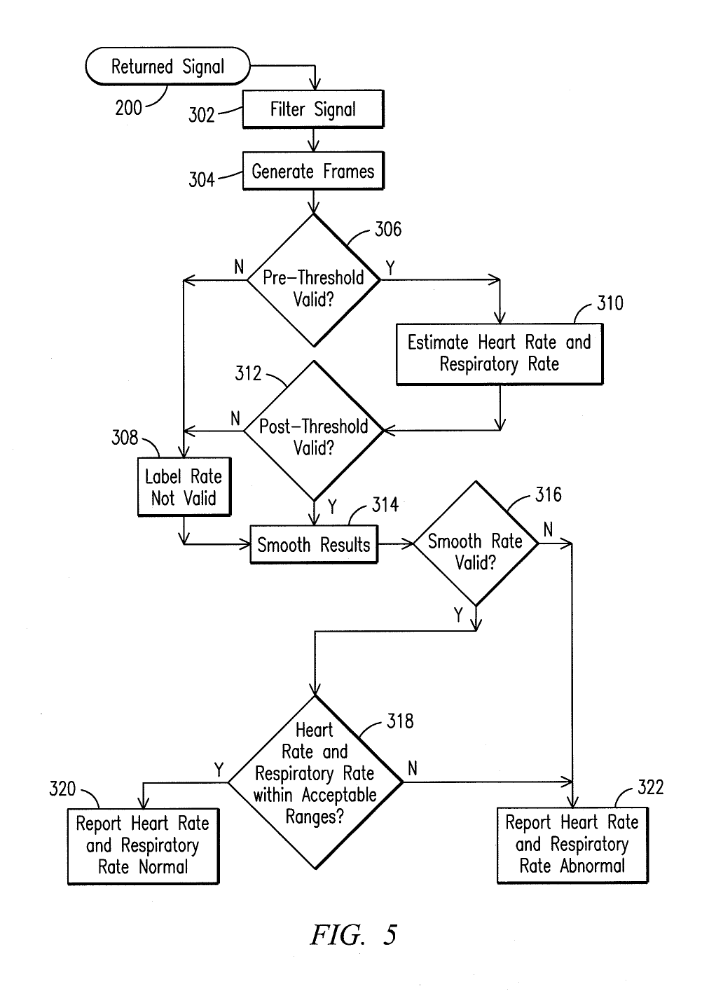

As can be seen in FIG. 5, rate estimation algorithms processes the returned signal 200, but in a different manner than the state estimation module. The rate estimation module in one example runs constantly in the background. Alternatively the rate estimation module operations on a computing device are dormant until called upon by the state estimation in a further embodiment. If the rate estimation constantly runs, it is able to more quickly return rate estimates, but will consume more processor resources. Alternatively, if dormant until called upon, the processing is slower to return rate information, but will consume fewer processor resources. Should the rate estimation run constantly, rate estimates generated during periods of gross body motion are simply labeled as not valid.

For the rate estimation the returned signal is continuously fed into a filter that extracts heartbeat signals with frequencies responsive to the presence of a heartbeat, and respiration signals with frequencies responsive to the presence of respiration. Frequencies responsive to the presence of a heartbeat typically range from about 1 Hz to about 3 Hz. Frequencies responsive to the presence of respiration typically range from just above 0 Hz (DC) to about 1 Hz. Consequently, the returned signal should cover at least those frequencies, and the heartbeat rate signal will be of a signal comprising from about 1 Hz to about 3 Hz, and the respiration rate signal will be of a signal comprising from just above 0 Hz (DC) to about 1 Hz. Filters such as low pass and band pass filters may be utilized to extract the heartbeat frames and respiration frames from each signal frame.

In one embodiment the system processing includes pre-thresholding and/or post-thresholding of the physiological rates as further detailed herein. The pre-thresholding and post-thresholding processes the physiological rates and determines a subset of the physiological rate estimates that are inside an acceptable threshold range. Alternatively, the pre-thresholding and post-thresholding processes the physiological rates and determines a subset of the physiological rate estimates that are outside an acceptable threshold range. The rate estimation module in one example is configured to provide smoothed physiological rates by processing the subset of the physiological rate estimates that are inside the acceptable threshold range. Alternatively, the rate estimation module in one example is configured to provide smoothed physiological rates by ignoring the subset of the physiological rate estimates that are outside the acceptable threshold range. In yet another example, the smoothed physiological rates are considered outside an acceptable rate range if a size of the subset of the physiological rate estimates that is subject to smoothing is less than a validity subset threshold. The validity subset threshold refers to the amount of data required to make a proper determination. If the size of the data subset is too small, the processing could be inaccurate and/or inconclusive.

Referring again to FIG. 5, the returned signals 200 in this example are filtered in a filtering step 302, and sampled to extract frames from the signal frame in a framing step 304. Heartbeat frames of a heartbeat frame duration are sampled from the heart rate signal at a heart rate sample rate. Respiration frames of a respiration frame duration are sampled from the respiration rate signal at a respiration rate sample rate. Frame samples are of a limited duration in terms of time. In one example, the frame samples cover limited periods of time such as ten seconds or thirty seconds. Heart rates are typically higher than respiration rates, and thus heartbeat frame durations are generally shorter than respiration frame durations. For example, a heartbeat frame duration of ten seconds or more have been found to provide sufficient information from which a heart rate can be estimated, while respiration frame durations of thirty seconds or more have been found sufficient.

The rate estimation algorithm in one example then pre-thresholds each heartbeat frame for suitability for further analysis to ascertain if the pre-threshold is valid, such as within an acceptable threshold range, in a pre-threshold validation step 306. During pre-thresholding, the rate estimation algorithm in one example checks the standard deviation or variance of the signal information in the heartbeat frame. Low values for either the standard deviation or variance indicates either an empty room, or noise, and the heart rate estimate for this frame would be labeled as not valid 308. High values for either the standard deviation or variance indicates motion and the heart rate estimate for this frame would be labeled as not valid 308. Threshold values for variance and standard deviation can be preprogrammed, user-adjustable, and/or adjusted by the algorithms themselves. Likewise the rate estimation algorithm pre-thresholds each respiration frame for suitability for further analysis, with threshold values similarly derived. The heart rate and respiration rate algorithms are typically not considered reliable during periods of motion, and thus the frame would be labeled as not valid 308 if motion is indicated. Otherwise, the frames are considered valid. This step of pre-thresholding 306 has been observed to reduce the heart rate estimation errors as well as respiration rate estimation errors.

The valid heartbeat frames are then processed through the rate estimation core algorithm(s). Various approaches can be employed, including spectral techniques. The algorithms may employ several techniques to reach a rate, such as: region of interest in magnitude squared FFT; peak in magnitude FFT; and peak in autocorrelation spectrum. Heart rate algorithms then estimate a heart rate for the frame in a rate estimation step 310. Likewise, respiration frames are processed and respiration rate algorithms estimate a respiration rate. In instances where the RADAR being used is not able to discern direction of motion, a heartbeat or a respiration may appear as two events. In such a case where this harmonic or “doublet-relation” exists, the algorithm reports the fundamental or lowest frequency, regardless of which frequency has the stronger peak.

In a further embodiment, after rate estimation 310, each heart rate estimate is subjected to a post-thresholding step 312 where the signal-to-noise ratio of the rate estimate is determined in the spectral domain and considered valid data if within an acceptable threshold range. In one example, if the signal-to-noise estimate is too low the algorithm labels the frame as not valid 308. Likewise, each respiration rate estimate is subjected to this post-thresholding step 312. Threshold values for the signal to noise ratio can be preprogrammed, user-adjustable, and adjusted by the algorithms themselves. Post-thresholding 312 has also been shown to reduce heart rate estimation error rates.

Once a heart rate and respiration rate have been estimated 310, and are within the post-threshold range, the estimated rates in this example are combined with other similar estimated rates, and the respective rates are smoothed in a smoothing step 314. Smoothing may be accomplished using techniques known in the art. Moving average or median filtering may be used in one embodiment. In one exemplary processing, frames labeled as not-valid are excluded from the smoothing operation and only the subset of valid frames are processed. In one example, if the number of valid frames in the subset is less that a validity subset threshold, the smoothed physiological rates are considered outside an acceptable rate range. The validity subset threshold in one example is approximately fifty percent or greater; while in another example, the validity subset threshold can be less that fifty percent for certain applications. For example, if too many frames are labeled not-valid, the smoothed rate estimation is also labeled not-valid in a smoothing rate validation step 316. For frames with valid smoothed rate estimation 316, the smoothed rate is compared with predetermined thresholds to assess whether the rate in within the acceptable range in a heart rate and respiration rate comparison step 318. The rate estimation algorithm will report the heart rate as abnormal 322 if it is outside its acceptable range or if the smoothed heart rate is labeled not-valid. The processing also reports the respiration rate as abnormal if it is outside its acceptable range or if the smoothed respiration rate is labeled not-valid. If the smoothed rate is within the range for the heart rate and respiratory rate, and not otherwise labeled as not-valid, the processing reports that the heart rate and respiratory rate are normal 320.

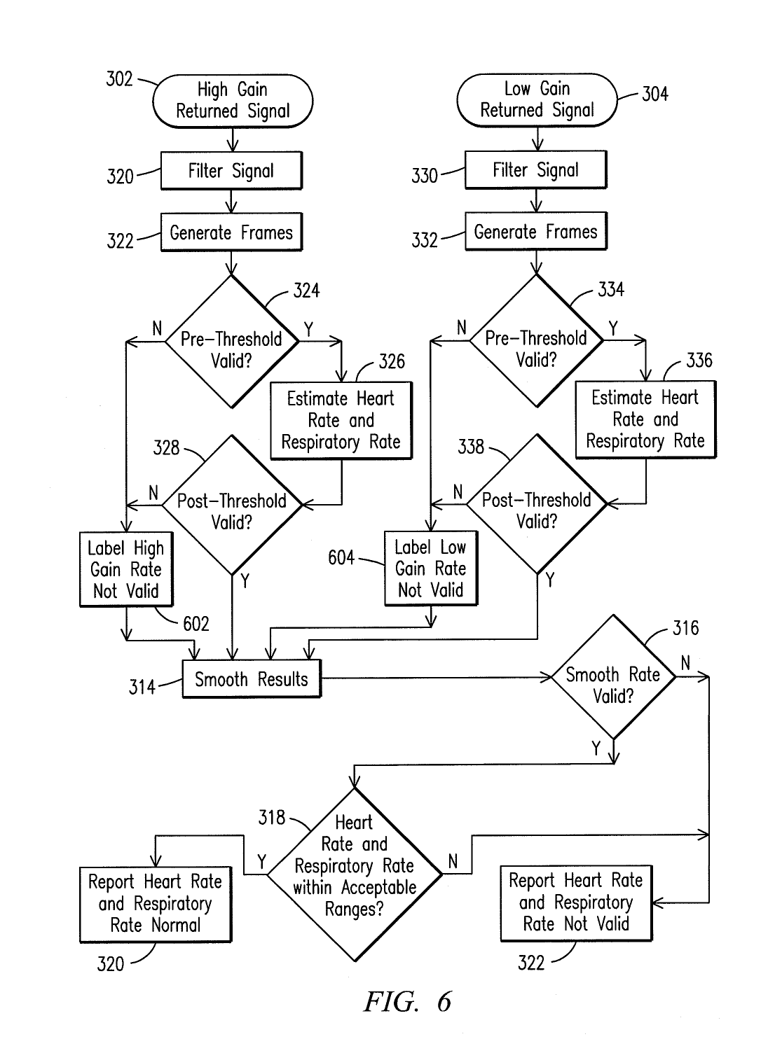

As depicted in FIG. 6, the returned signal may comprise a high gain returned signal 302 and a low gain returned signal 304. In one embodiment each signal is processed individually for rate estimations and the heart rate results from both channels and are considered in the heart rate smoothing function, and the respiration rate results from both channels are considered in the respiration rate smoothing function. For example, the high gain signal 302 may be filtered in a high gain filtering step 320, framed in a high gain frame generating step 322, pre-thresholded in a high gain pre-thresholding step 324. A heart rate and respiration rate are estimated in a high gain heart rate and respiration rate estimation step 326 and the results are subject to post-thresholding in a high gain post-thresholding step 328. If the high gain post-thresholding step 328 indicates that the results are not valid, then the results are labeled not valid in step 602. The results are then considered by the smoothing function 314. The low gain signal 304 is also separately filtered in a low gain filtering step 330, framed in a low gain framing step 332, pre-thresholded in a low gain pre-thresholding step 334, wherein a heart rate and respiration rate are estimated in a low gain heart rate and respiration rate estimation step 336. The results are post-thresholded in a low gain post-thresholding step 338, and those results are also considered by the smoothing function 314. If the low gain post-thresholding step 338 indicates the results are not valid, then the results are labeled not valid in step 604. Essentially, the smoothing function has more heart rate and respiration rate estimates to consider when two signals being processed instead of just one. As discussed above the smoothed results are checked for validity in a smoothing rate validation step 316. In an embodiment, if too many frames are labeled not-valid, the smoothed rate estimation is also labeled not-valid in the smoothing rate validation step 316. For frames with valid smoothed rate estimation 316, the smoothed rate is compared with predetermined thresholds to assess whether the rate in within the acceptable range in a heart rate and respiration rate comparison step 318. The rate estimation algorithm will report the heart rate as abnormal 322 if it is outside its acceptable range or if the smoothed heart rate is labeled not-valid. The processing also reports the respiration rate as abnormal if it is outside its acceptable range or if the smoothed respiration rate is labeled not-valid. If the smoothed rate is within the range for the heart rate and respiratory rate, and not otherwise labeled as not-valid, the processing reports that the heart rate and respiratory rate are normal 320

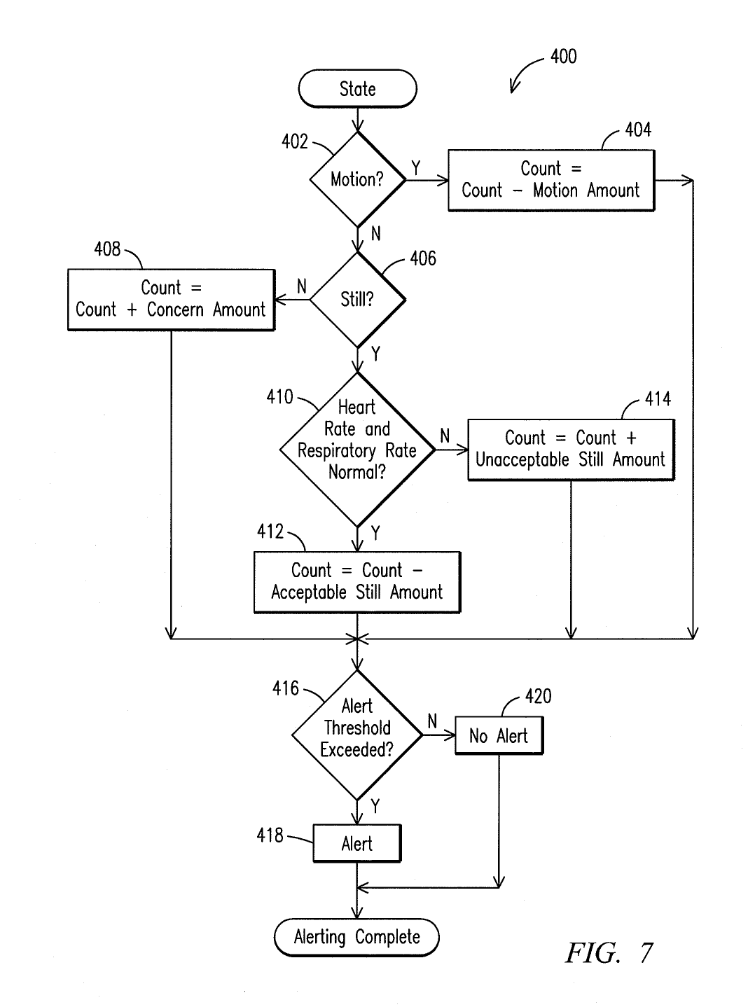

FIG.7 shows a flow chart for the alerting module 400 according to one embodiment. The state estimates are first checked for ‘motion’ states in a motion checking step 402. If the state is determined to be ‘motion’, the count is decreased by a predetermined motion amount 404 since an observation of ‘motion’ is generally considered good health. If the current value of the counter is less than the predetermined motion amount, the count decreaser in 404 sets the counter to zero to prevent long periods of motion to overshadow recent conditions that may warrant an alert. If the state is determined to not be in ‘motion’ in the motion checking step 402, the state is checked for ‘still’ states in a still checking step 406. If the state is determined to not be ‘still’ in 406 (i.e. that state is ‘concern’) the count is increased by a predetermined concern amount in 408 since an observation of ‘concern’ is generally considered poor health. If the state is determined to be ‘still’ in the still checking step 406, the heart rate and respiration rates are checked for normal in a heart rate and respiration rate checking step 410. If the heart rate and respiration rate are normal in 410, the count is decreased by a predetermined acceptable still amount in 412 since an observation of normal heart rate and respiration rate is generally considered good health. If the current value of the counter is less than the predetermined acceptable still amount, the count decreaser in 412 will set the counter to zero to prevent long periods of normal heart rate and respiration rate to overshadow recent conditions that may warrant an alert. If either the heart rate or respiration rate is not normal in 410, the count is increased by a predetermined unacceptable still amount in 414 since an observation of abnormal heart rate and respiration rate is generally considered poor health. After all incrementing or decrementing of the count, the count is compared to an alert threshold in an alert threshold comparison step 416. If the count exceeds the alert threshold, an alert 418 is generated. If the count does not exceed the alert threshold, no alert is generated 420. Various state estimates may each have its own adjustment value, and that amount may be an increment or a decrement to the predetermined alert threshold. Furthermore, the adjustment values may be the same or different, and may be adjusted as the systems learns a subject.

Various alerting algorithms can be used to determine whether or not to generate an alert. A simple algorithm may keep a count based on the estimated states, and that count can be monitored to see if it exceeds a predetermined threshold. Similarly, the amount of time a certain state is estimated can be set as a threshold. For example, if a majority, or all of the state estimates are set to concern state during a certain time period, such as three minutes, then the alert may be “sounded.” This allows the system to ride-through or gives low weight to transient periods of one nature in favor of a trend of another nature. As a result, the alert in this example is not sounded for every concern state, which may lend credence to alerts that are generated. Alerting algorithms in the alert module may also be learning algorithms that learn the subject being monitored, for example through feedback regarding earlier alerts.

In one embodiment the alert module may automatically turn off if the alert condition is not maintained. For instance, if after exceeding the alert count threshold, motion or acceptable physiological parameters and acceptable physiological rates are detected, the alert count may be reduced below the alert count threshold. In another embodiment, the alert module will remain in an alert state until an operator manually intervenes to reset the alert criteria.

The alerting algorithm in other examples also considers objective information about the subject. For example, objective data about a heart rate, respiration rate, and/or related trends (i.e. rates of change of heart or respiration rates, or inter-relationships of the two etc) of persons of a similar sex, and age may be used as criteria against which the subject being monitored is measured. In addition, subjective criteria about the specific individual being monitored may be used. If the subject is known to have heart problems, lung problems, sleep apnea, or other health conditions that may warrant adjustments to the acceptable heart rate, respiration rate, and/or whatever other trends the algorithms may monitor, the algorithm can account for that. Further subjective criteria may include psychological factors. For example, is the subject is in a heightened state of anxiety the algorithms may adjust for that by expecting different heart rates and/or respiration rates. If the subject is a suicide risk, the system may alert sooner rather than later. The alerting algorithm may also consider environmental factors that might influence a heart rate or respiration rate, such as a room temperature, or external threats.

In one embodiment, an adjustment value is assigned to each state estimation. For example, a motion state may be assigned a −1, a concern state may be assigned a +1, a still state with heart and respiration rates within acceptable ranges may be assigned a −1, and a still state with either a heart or respiration rate outside its respective acceptable range may be assigned a +1. A count may be maintained with a minimum value, and a threshold value. For each time a motion state is estimated, the count would decrease by 1. For each time a concern state is estimated, the count would increase by 1. The threshold would be set such that excessive estimates of negative health states (i.e. concern or still state with either a heart or respiration rate outside its respective acceptable range) would cause the count to exceed the threshold, and an alert would be triggered. Different adjustment amounts could be applied, and the algorithm could consider the count and durations in the alert analysis. For example, if a minimum percentage of bad health states are estimated during a given time period, an alert may be triggered. In another embodiment, the system automatically adjusts parameter settings such as the alert count threshold based on learning from past experience and historical data where the alert count increases above and below a current alert count threshold within a limited time period.

The algorithms employed in each of the state, rate, and alerting modules may learn through various ways. For example, the system may prompt an operator for feedback once an alert has been generated. If the feedback indicates many false alerts, the algorithms may adjust accordingly. Further, the algorithms may initiate questions for the operator about the state of the subject. Alternately, the operator may periodically tell the system the state of the subject and the system can compare its instant estimates with the information fed to it.

One embodiment of the present system provides an inexpensive, low complexity system for monitoring a subject's vital signs. This innovative design makes monitoring available to those who were unable to afford such systems because the system is more affordable, and less complex. The system is so much less complex that the monitoring system may be a cell phone. Using a cell phone would make the alerting easier because the cell phone itself could call the person that needs to be alerted. Existing cell phones used for communication could have additional hardware inside, such as the RADAR circuit boards. The advantage of such a system is readily apparent, and could enable individuals to be monitored full time, yet not be restricted in their activities. As a result, the system disclosed herein provides a significant improvement over the existing systems and fulfills a long felt need in the art.

It should be understood that the inventive system and method disclosed herein may be implemented in any appropriate operating system environment using any appropriate programming language or programming technique. The system can take the form of a hardware embodiment, a software embodiment or an embodiment containing both hardware and software elements. In one embodiment, the system is implemented in software (controls) and hardware (sensors), which includes but is not limited to firmware, resident software, microcode, etc. Furthermore, parts of the system can take the form of a computer program product accessible from a computer-usable or computer-readable medium providing program code for use by or in connection with a computer or any instruction execution system. Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD. The display may be a tablet, flat panel display, PDA, or the like.

In one embodiment, multiple RADAR sensors are combined to give better coverage of the physical space and detection of motion and physiological parameters. The state estimation and rate estimation modules can be expanded to assign states and estimate rates based on data from the plurality of signals received. In one embodiment, a RADAR unit may be mounted from a ceiling and a second RADAR unit may be mounted on a wall. In another embodiment, multiple RADAR units may be mounted from a ceiling in a grid pattern to provide adequate coverage for a large room.

In one embodiment, several RADAR sensors are linked with a processing system to monitor multiple subjects such as multiple rooms in a nursing home or multiple cells in a prison environment. The processing system will uniquely identify and track the separate signals in order to perform the state estimation, rate estimation and alerting on each separate subject's data stream from the RADAR devices.

A data processing system suitable for storing and/or executing program code will include in one example at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. Input/output or I/O devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

While various embodiments of the present invention have been shown and described herein, it will be apparent that such embodiments are provided by way of example only. Numerous variations, changes and substitutions may be made without departing from the invention herein. Accordingly, it is intended that the invention be limited only by the spirit and scope of the appended claims.

The invention claimed is:

1. A system for monitoring physiology, comprising:
a RADAR apparatus comprising a RADAR transmitter configured to deliver a RADAR signal to a subject, and a RADAR receiver configured to receive a returned RADAR signal from the subject;
a state estimation module configured to process the returned signal to detect a presence of motion and set a motion state upon said presence of motion, said state estimation module configured to detect a presence of one or more physiological parameters, said physiological parameters comprising at least one of heartbeat and respiration, and said state estimation module configured to assign a still state or a concern state based on said presence of physiological parameters;
a rate estimation module configured to process the returned signal and estimate one or more estimated physiological rates comprising at least one of an estimated respiration rate and an estimated heart rate; and
an alerting module configured to provide an alert if an alert value exceeds an alert value threshold, wherein the alert value is derived from at least one of the motion state, concern state, still state and the estimated physiological rates.

2. The system of claim 1, wherein the state estimation module only detects the presence of physiological parameters when there is no presence of motion.

3. The system of claim 1, wherein the rate estimation module only provides the estimated physiological rates when there is the presence of one or more physiological parameters

4. The system of claim 1, wherein the state estimation module is configured to
set the motion state if there is the presence of motion,
set the still state if there is no presence of motion and there is presence of at least one of the physiological parameters,
otherwise, set the concern state.

5. The system of claim 1, wherein the state estimation module is configured to set the concern state if there is no presence of at least one of the physiological parameters.

6. The system of claim 1, wherein the state estimation module is configured to set the still state is set if there is no presence of motion and the estimated physiological rates are within an acceptable rate range.

7. The system of claim 1, wherein the state estimation module is configured to set the concern state is set if there is no presence of motion and at least one of the estimated physiological rates are outside an acceptable rate range.

8. The system of claim 1, wherein the returned signal comprises at least a first and a second signal, the first and the second signal comprising signals having different gain characteristics, wherein the first signal is processed for the presence of motion and the second signal is processed for the presence of at least one of said physiological parameters, wherein detection of the presence of motion results in setting the motion state, and wherein concern state is set if there is no presence of motion and no presence of at least one of said physiological parameters.

9. The system of claim 1, wherein the returned signal comprises at least a first and a second signal, the first and the second signal comprising signals having different bands, wherein the first signal is processed for the presence of motion and the second signal is processed for the presence of at least one of said physiological parameters, wherein detection of the presence of motion results in setting the motion state, and wherein the concern state is set if there is no presence of motion and no presence of at least one of said physiological parameters.

10. The system of claim 9, wherein the returned signal further comprises a third signal, the third signal having different band from said first and second signals, wherein the third signal is processed for at least one of said physiological parameters.

11. The system of claim 1 where the state estimation module is configured to perform state estimation of the returned signal based upon statistical features, spectral features, temporal features or combinations thereof.

12. The system of claim 1, wherein the rate estimation module is configured to perform at least one of pre-thresholding and post-thresholding of the estimated physiological rates.

13. The system of claim 12, wherein at least one of the pre-thresholding and post-thresholding determines a subset of the estimated physiological rates that are within an acceptable threshold range.

14. The system of claim 13, wherein the rate estimation module is configured to provide smoothed physiological rates by processing the subset of the estimated physiological rates.

15. The system of claim 14, wherein the smoothed physiological rates are considered outside an acceptable rate range if a size of the subset of the estimated physiological rates are less than a validity subset threshold.

16. The system of claim 1, wherein the alert value decreases upon setting the motion state or upon setting the still state and having estimated physiological rates that are within acceptable rate ranges; and said alert value increases if setting the concern state or upon setting the still state and having estimated physiological rates that are outside acceptable rate ranges.

17. The system of claim 1, further comprising at least one additional RADAR transmitter to deliver at least one additional RADAR signal to at least one additional subject, and at least one additional RADAR receiver to receive returned RADAR signals from the additional subject.

18. A system for monitoring physiology, comprising:
a RADAR apparatus comprising a RADAR transmitter configured to deliver a RADAR signal to a subject, and a RADAR receiver configured to receive a returned RADAR signal from the subject, wherein the returned signal comprises at least a first signal and a second signal, each having different signal characteristics;
a state estimation module configured to process at least the first signal and the second signal to detect a presence of motion and one or more physiological parameters, said physiological parameters comprising at least one of heartbeat and respiration, wherein the state estimation module is configured to assign a state estimation state based on the presence of motion, the physiological parameters, and combinations thereof;
a rate estimation module configured to further process at least the first signal and the second signal and provide estimated physiological rates comprising at least one of a heart rate and a respiration rate; and
an alerting module configured to set an alert value and communicate an alert based on the alert value, wherein the alert value is derived from processing from the state estimation module and the rate estimation module.

19. The system of claim 18, wherein the state estimation module is configured to set the state estimation state to motion state if there is presence of motion; set the state estimation state to concern state if there is no presence of motion and there are no physiological parameters detected; set the state estimation state to still state if there is no presence of motion but at least one of the estimated physiological rates are within an acceptable rate range; and otherwise set the state estimation state to concern state.

20. The system of claim 18, wherein the rate estimation module only processes estimated physiological rates if there is no presence of motion and at least one of the physiological parameters are detected.

21. The system of claim 18, wherein the state estimation module is configured to detect heartbeat and respiration, and if there is no presence of motion and there is no heartbeat or respiration, the state estimation state is set to concern state; if there is no presence of motion, there is at least one of heartbeat or respiration, and at least one of the estimated physiological rates are within an acceptable range, the state estimation state is set to still state, and otherwise the state estimation state is set to concern state.

22. The system of claim 18, wherein the rate estimation module is configured to estimate heart rate and respiration rate, wherein the rate estimation module is configured to set the state estimation state to still state if the heart rate and the respiration rate are within an acceptable range, otherwise to set the state estimation state to concern state.

23. The system of claim 18, wherein the rate estimation module is further configured to perform at least one of pre-thresholding and post-thresholding of the first signal, the second signal, or both the first signal and the second signal.

24. The system of claim 23, wherein at least one of pre-thresholding and post-thresholding determines a subset of the estimated physiological rates that are inside an acceptable threshold range.

25. The system of claim 24, wherein the rate estimation module is configured to provide smoothed physiological rates by processing the subset of the estimated physiological rates.

26. The system of claim 25, wherein the smoothed physiological rates are considered outside an acceptable rate range if a size of the subset of the estimated physiological rates is less than a validity subset threshold.

27. The system of claim 18, wherein the alert value decreases upon setting the motion state or upon setting the still state and having estimated physiological rates that are acceptable; and said alert value increases if setting the concern state or upon setting the still state and having estimated physiological rates that are unacceptable.

28. A method for monitoring physiology, comprising:
providing a RADAR transmitter to deliver a RADAR signal to a subject, and a RADAR receiver to receive a returned RADAR signal from the subject;
processing the returned RADAR signal to detect a presence of motion;
based upon the presence of motion, further processing the returned RADAR signal to determine a presence of physiological parameters, said physiological parameters comprising at least one of heartbeat and respiration;
based upon the presence of the physiological parameters, processing estimated physiological rates from the returned RADAR signal, said estimated physiological rates comprising at least one of a heart rate and a respiration rate; and
setting an alert based on at least one of the presence of motion, the presence of physiological parameters and the estimated physiological rates.

29. The method of claim 28, wherein the determination of the presence of physiological parameters is performed only when there is no presence of motion.

30. The method of claim 28, wherein the processing of the estimated physiological rates is performed only when the presence of physiological parameters are detected.

31. The method of claim 28, comprising setting a concern state if there is no presence of motion and there is no presence of physiological parameters; setting a still state if there is no presence of motion and each of the estimated physiological rates is within a respective acceptable rate range; and otherwise setting a concern state.

32. The method of claim 28, wherein the returned signal is further processed for both heartbeat and respiration, and wherein both heart rate and respiration rate are estimated, further comprising setting a still state if both the heartbeat and the respiration are detected and both the estimated rates are within respective acceptable ranges.

33. The method of claim 28, wherein the returned signal comprises a first signal and a second signal having different gain characteristics.

34. A non-transitory tangible computer-readable medium having computer-executable instructions for performing the steps recited in claim 28.
